(19) United States
(12) Patent Application Publication
(10) Pub. No.: US 2006/0053850 A1
(43) Pub. Date: Mar. 16, 2006

(54) Package for storing a formulate liquid inoculating solution containing rhizobia and process for growing rhizobia in it

(75) Inventors: Eliana G. M. Lemos, Sao Paulo (BR); Lucia Maria Carareto Alves, Sao Paulo (BR); Roberto Parducci Camargo, Sao Paulo (BR); Salvador Parducci, Sao Paulo (BR)

Correspondence Address: OSTROLENK FABER GERB & SOFFEN, 1180 AVENUE OF THE AMERICAS, NEW YORK, NY, 100368403, US

(73) Assignee: Bioarts Industria E Comercio de Biotechnologia Ltda.

(21) Appl. No.: 11/224,659

(22) Filed: Sep. 12, 2005

(30) Foreign Application Priority Data
Sep. 14, 2004 (BR) 0,404,749-4

(51) Int. Cl. C12M 3/00

(52) U.S. Cl. 71/7; 435/289.1

(57) ABSTRACT

A package for storing a formulated liquid inoculating solution containing rhizobia and process for growing rhizobia in it, wherein the package is comprised of a bag with layer of flexible PVC or yet a simple or double layer of PEBD (Low Density Polyethylene Film) provided with a plastic beak with a silicone rubber. The process allows the inoculation step with stocks of rhizobia recommended in the nutritive solution originally contained within the plastic bags, followed by allowing growing of bacteria to occur in its own package, discarding the use of fermenters to promote the growing of colonies up to at least 10⁸.

### BACKGROUND OF THE INVENTION

The present invention relates to a package for storing the formulated liquid inoculating solution containing rhizobia and a process of growing rhizobia in it. More particularly, the invention refers to a package for packing and storing rhizobia in solutions of distinguished liquid formulation, being this package defined by a bag with volume ranging of 200, 500, 1000, 2000 and 4000 ml.

As it is known by those skilled in the art, packages usually used for packing rhizobia are constituted of bags with 100, 150 and 300 ml that are provided with an inoculating with the total bacteria in an already grown condition.

Although it is widely used, this solution shows as an inconvenience the fact that it has a high industrial cost of manufacturing inoculating, because it is necessary to use fermenters in sterile areas.

Another inconvenience relates to the rhizobia survival degree, because they are inserted in the solution when they are already grown, what reduces the validity time for effectively using the packaged liquid solution.

### SUMMARY OF THE INVENTION

It is one object of the invention to provide a package to store the formulated liquid inoculating solution containing rhizobia that presents a low industrial cost for manufacturing the inoculating, once it is not required to use fermenters.

It is another object of the invention to provide a package to store a formulated liquid inoculating solution containing rhizobia that allows a further introduction of stocks of bacteria (rhizobia) recommended in the nutritive solution inside the package.

It is another object of the present invention to provide a process of growing rhizobia in its own package that increases the lifetime of the solution, because the stocks of bacteria are inserted in the package in a further step that promotes the packaging of the package. This process allows the stocks of bacteria introduced in the package in the sites next to where they are used, assuring a number of viable cells per milliliter to be used in the inoculation of leguminous plants, without the risk of rhizobia losing its capability to infect the roots, because of the temperature during transportation from the facility plant to the place where it will be used. This process avoids returning inoculating with validity time expired, resulting from the low number of viable cells. This returning of the product back to the industries, estimated in 30% out of the commercialized total, represents a significant percent of the cost of the product (delivery and return freight, taxes and costs to eliminate the inoculating organic dejects with low viable cells rates without causing environmental impact) and which is eliminated with the present invention, because only distinguished nutritive broth will be put in the sites next to consumption (resellers, cooperatives and large consumers), being the stocks of bacteria introduced 15 a 45 days before being used, in the same sites, which assures quality to all users without burdening the product price with the returning component.

### BRIEF DESCRIPTION OF THE DRAWINGS

The present invention shall be described as follows with reference to the amended figures, wherein:

FIG. 1 shows a picture of a package for storing a formulated liquid inoculating solution containing rhizobia in a condition of maximum volume capacity;

FIG. 2 shows a picture of a package for storing a formulated liquid inoculating solution containing rhizobia in an empty and open condition, with its stopper shown beside and next to the filling beak used to insert the liquid or solution still exempt of rhizobia; and

FIG. 3 shows a graph of the average number of colonies found in the package containing the differentiated culture broth.

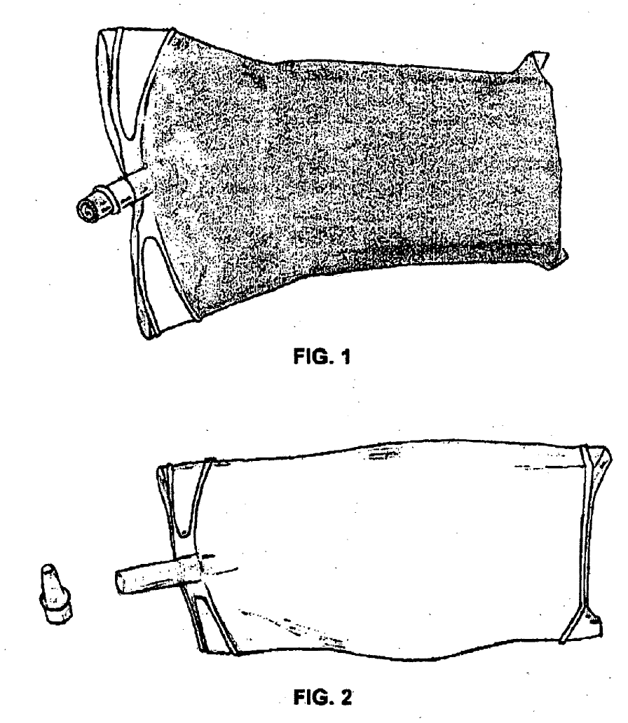

### DETAILED DESCRIPTION OF PREFERRED EMBODIMENTS OF THE INVENTION

According to those illustrations, the package for storing a formulated liquid inoculating solution containing rhizobia, object of the present invention, is comprised of a bag with a simple layer of flexible PVC or yet a simple or double layer of PEBD (Low Density Polyethylene Film) that allows the gas exchanges of the bacteria with the environment and the maintenance of the bacterial viability for at least 9 months.

These packages have a plastic beak provided with a silicone rubber, which allows the introduction of a steel needle, not shown, to allow the inoculation step with stocks of rhizobia recommended in the nutritive solution originally contained inside the plastic bags.

This device allows growing of bacteria to occur in its own package, discarding the use of fermenters to promote the colony growing until 10⁸, at least.

It is also part of the invention a process for growing rhizobia in its own package, which consists of further inoculating stocks of rhizobia through a needle that trespass the stopper of a package already filled with nutritive solution.

This process presents the advantages of assuring the fermentation step to occur inside the bag itself containing the differentiated culture broth, previously sterilized. This situation exempts the use of fermenters in aseptic industrial plants.

Preferably, this culture broth contains a source of carbon (manitol, glycerol or glucose), different sugars responsible for maintaining the integrity of the bacterial cellular walls (manitol, glucose, arabinose, mannose, glycerol), an aggregator agent as a polyvinylpirrolidone or starch, a source of nitrogen (yeast extract), salts (magnesium and sodium chloride, phosphates) and an source of iron.

The medium pH ranges from 6 to 8 and the growing pH ranges from 28 to 31° C. After obtaining the adequate number of bacteria in the medium of growing the stocks recommended undergo a quality control.

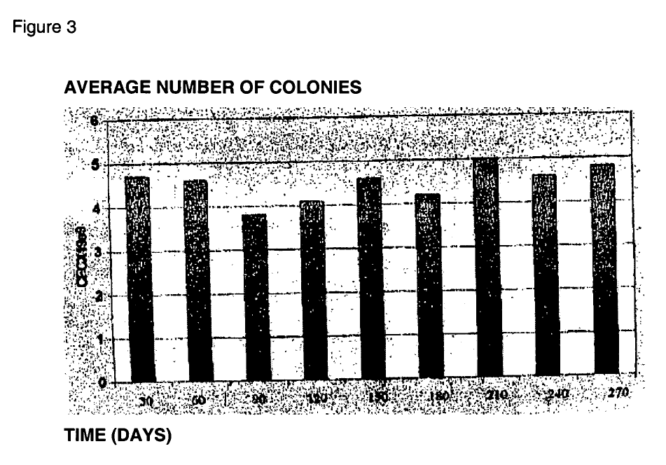

Thus, the liquid formulation presents more than 10⁹ bacteria per milliliter of inoculating during 8 months consecutively, as shown in FIG. 3.

With this procedure, it is possible to protect a type of package for storing the liquid inoculating solution and the formulation, besides to assure that the process of fermentation (growing of nitrogen-fixing bacteria) takes place within the solution already packaged. This process allows a greater resistance and survival of the microorganisms within the packages as well as in the seeds and in the soil after its application in the planting furrow.

Survival essays showed that they can survive up to 10 months within the product without losing its biological capability.

Although a preferred inventive concept was above described changes are possible and performable within the scope of the present invention.

What is claimed is:

1. A package for storing a formulated liquid inoculating solution containing rhizobia, wherein the package is comprised of a bag with layer of flexible PVC or yet a simple or double layer of PEBD (Low Density Polyethylene Film) provided with a plastic beak with a silicone rubber.

2. The package for storing a formulated liquid inoculating solution containing rhizobia in accordance with claim 1, wherein the package is defined by a bag with volume ranging of 200, 500, 1000, 2000, and 4000 ml.

3. A process of growing rhizobia in its own package, wherein it allows the inoculation step with stocks of rhizobia recommended in the nutritive solution originally contained within the plastic bags, followed by allowing growing of bacteria to occur in its own package, discarding the use of fermenters to promote the growing of colonies up to at least 10⁸.

4. The process of growing rhizobia in its own package in accordance with claim 3, wherein it consists of further inoculating stocks of rhizobia through a needle that trespasses the stopper of the package already filled with a nutritive solution.
